(19) United States
(12) Patent Application Publication
(10) Pub. No.: US 2006/0020212 A1
(43) Pub. Date: Jan. 26, 2006

(54) Portable vein locating device

(76) Inventors: Tianning Xu, San Antonio, TX (US); Yufang Dai, San Antonio, TX (US)

Correspondence Address: WAYNE J COLTON INC;THE MILAM BUILDING SUITE 1032, 115 EAST TRAVIS STREET, SAN ANTONIO, TX, 78205, US

(21) Appl. No.: 10/899,518

(22) Filed: Jul. 26, 2004

(51) Int. Cl. A61B 6/00

(52) U.S. Cl. 600/473

(57) ABSTRACT

A portable vein locating device includes one or more infrared illuminators for transmission through a patient's skin of infrared light and a vein imaging module for determining the location of a vein beneath the skin by detecting the absence of backscattered infrared light. As the device is passed over a patient's skin, infrared light from the infrared illuminators penetrates through the patient's skin and is absorbed by veins under the skin but scattered by the fat and other tissues surrounding the veins. Backscattered infrared light from the areas about the veins is detected by the vein imaging module, which includes a number of infrared light detectors, for detecting the backscattered light, and a display, for indicating to the medical caregiver the location beneath the skin of detected veins.

### FIELD OF THE INVENTION

The present invention relates to medical care. More particularly, the invention relates to a portable vein locating device for aiding a medical care giver in locating a vein through the skin of a patient, especially an obese patient or a patient with dark skin color.

### BACKGROUND OF THE INVENTION

In the case of an obese and/or dark skinned patient, it is often very difficult for a medical caregiver to locate a vein beneath the patient's skin. To this end, various devices have been proposed for facilitating vein location. In particular, a number of devices have been proposed for illuminating the patient's skin with ambient visible light or an infrared light source and viewing the result through a camera. Unfortunately, such previously proposed devices are generally bulky and expensive in implementation. Additionally, such devices suffer from reduced contrast due to surface reflection and are sensitive to the ambient lighting conditions.

With the deficiencies of the prior art in mind, it is an overriding object of the present invention to improve of the prior art by providing a method utilizing a small and inexpensive device for locating beneath a patient's skin of a vein. Additionally, it is an object of the present invention to provide such a device that provides high resolution and is not sensitive to ambient lighting conditions.

### SUMMARY OF THE INVENTION

In accordance with the foregoing objects, the present invention—a portable vein locating device—generally comprises one or more infrared illuminators for transmission through a patient's skin of infrared light and a vein imaging module for determining the location of a vein beneath the skin by detecting the absence of backscattered infrared light. The portable vein locating device is utilized by gently caressing a patient's skin in the area in which a vein is to be located with the portion of the device containing the infrared illuminators and vein imaging module. As the device is passed over the skin, infrared light from the infrared illuminators will penetrate through the patient's skin and be generally absorbed by veins under the skin but scattered by the fat and other tissues surrounding the veins, which tissues are very translucent and structured in nature. Backscattered infrared light from the areas about the veins is then detected by the vein imaging module, which includes a plurality of infrared light detectors, for detecting the backscattered light, and a display, for indicating to the medical caregiver the location beneath the skin of detected veins.

Finally, many other features, objects and advantages of the present invention will be apparent to those of ordinary skill in the relevant arts, especially in light of the foregoing discussions and the following drawings, exemplary detailed description and appended claims.

### BRIEF DESCRIPTION OF THE DRAWINGS

Although the scope of the present invention is much broader than any particular embodiment, a detailed description of the preferred embodiment follows together with illustrative figures, wherein like reference numerals refer to like components, and wherein:

FIG. 1 shows, in a perspective view, the preferred embodiment—a generally ink pen-sized locating stick—of the portable vein locating device of the present invention;

FIG. 2 shows, in a perspective view, details of the infrared illuminators and vein imaging module as contained within the locating stick of FIG. 1;

FIG. 3 shows, in a cross-sectional view taken along cut line 3-3 of FIG. 1, various details of the infrared illuminators and imaging module depicted in FIG. 2;

FIG. 4 shows, in a perspective view, an alternate embodiment—a locating box—of the portable vein locating device of the present invention;

FIG. 5 shows, in a partially cutaway perspective view corresponding to FIG. 4, details of the infrared illuminators and camera as contained within the locating box of FIG. 4;

FIG. 6 shows, in a cross-sectional view taken along cut line 6-6 of FIG. 4, various details of the camera depicted in FIG. 5 and associated imaging components;

FIG. 7 shows, in a cross-sectional view taken along cut line 7-7 of FIG. 4, various details of the infrared illuminators depicted in FIG. 5; and

FIG. 8 shows, in a cross-sectional view taken along cut line 8-8 of FIG. 4, various details of the infrared illuminators and camera as depicted in FIG. 5.

### DETAILED DESCRIPTION OF THE PREFERRED EMBODIMENT

Although those of ordinary skill in the art will readily recognize many alternative embodiments, especially in light of the illustrations provided herein, this detailed description is exemplary of the preferred embodiment of the present invention, the scope of which is limited only by the claims appended hereto.

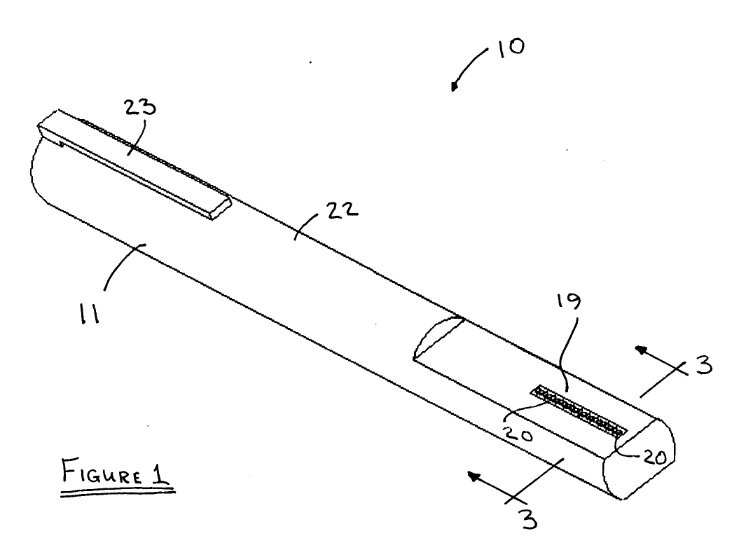

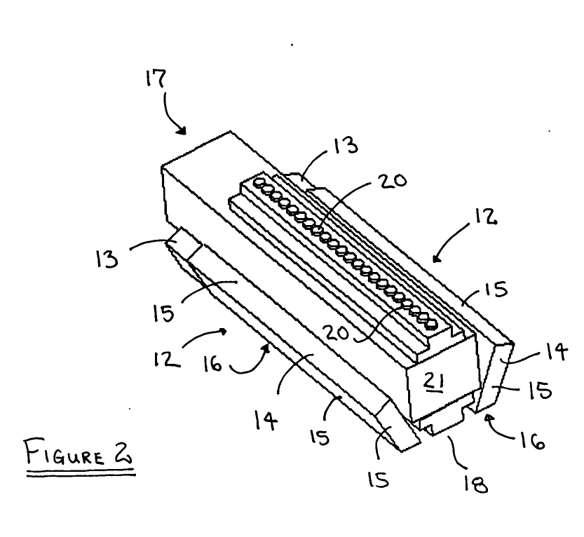

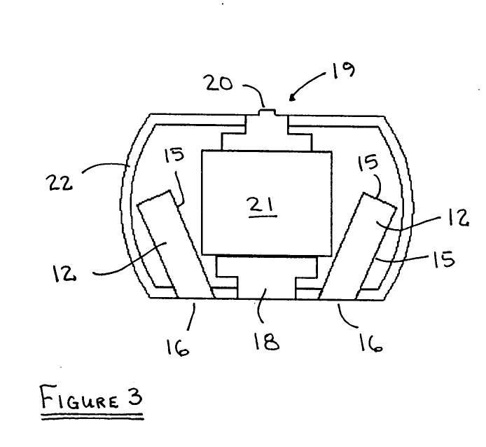

As shown in FIGS. 1 through 3, the portable vein locating device 10 of the present invention preferably comprises an ink pen-sized locating stick 11 having integrated therein one or more infrared illuminators 12 and a vein imaging module 17. As will be better understood further herein, the portable vein locating device 10 is utilized by gently caressing a patient's skin in the area in which a vein is to be located with the end of the locating stick 11 containing the infrared illuminators 12 and vein imaging module 17. Infrared light from the infrared illuminators 12 will penetrate through the patient's skin and be generally absorbed by veins under the skin but scattered by the fat and other tissues surrounding the veins, which tissues are very translucent and structured in nature. Backscattered infrared light from the areas about the veins is then detected by the vein imaging module 17, which includes a plurality of infrared light detectors 18, for detecting the backscattered light, and a display 19, for indicating to the medical caregiver the location beneath the skin of detected veins.

As shown in the figures, the infrared illuminators 12 of the locating stick 11 preferably each comprise an infrared light emitting diode (“LED”) 13 mated with a diffusing light guide 14. As will be appreciated by those of ordinary skill in the art, such a diffusing light guide 14 may be implemented by doping an optical material with aluminum powder or other dopants as is widely known in the manufacture of cellular telephone backlighting systems, television remote controls and the like. As also will be appreciated by those of ordinary skill in the art, the light guide 14 is manufactured with most sides 15 provided with a reflective coating and the sides 16 adjacent the infrared LED 13 and to be placed adjacent the patient's skin being substantially transparent to infrared light. In this manner, infrared light from the infrared LED 13 may directed through the patient's skin.

As also shown in the figures, the infrared light detectors 18 of the locating stick 18 are arranged in a generally linear array. Likewise, a plurality of LEDs 20, which form the display 19, are also arranged as a generally linear array corresponding to that of the light detectors 18, which, as will be appreciated by those of ordinary skill in the art, may comprise any of a variety of infrared sensitive optical sensors such as, for example, a charge coupled device (“CCD”) sensor array, a complementary metal oxide semiconductor (“CMOS”) sensor array, a phototransistor sensor array or the like. In any case, the LEDs 20 of the display 19 light to indicate the presence under the skin of a vein as detected by the elements of the infrared light detectors 18. As will be appreciated by those of ordinary skill in the art, an LED driver circuit 21, which is readily within the reach of those of ordinary skill in the art, is implemented to preferably light the LEDs 20 over the light detectors 18 that do not receive backscattered infrared light, thereby giving an intuitive indication of the location beneath the locating stick 11 of a vein.

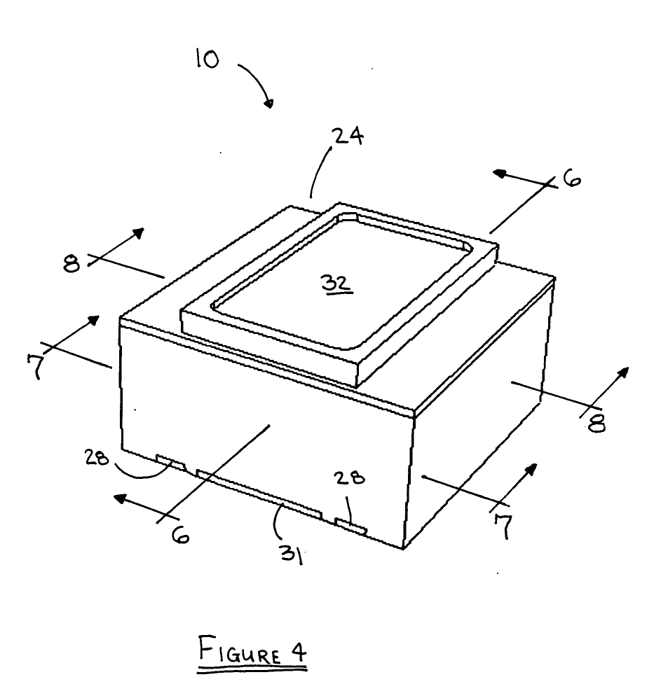

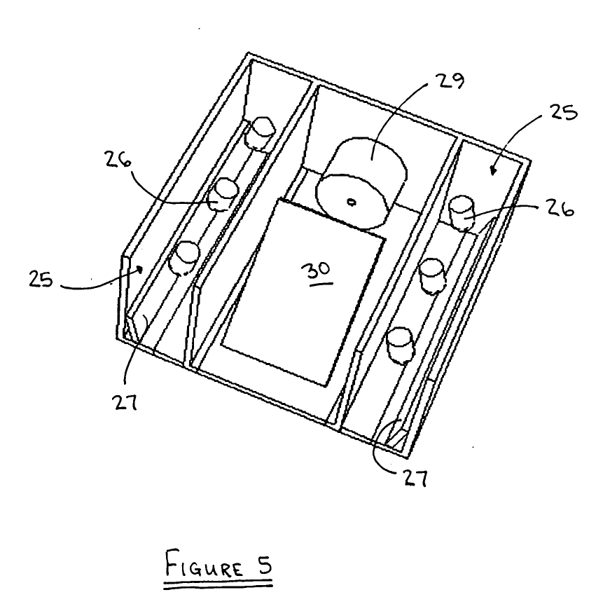

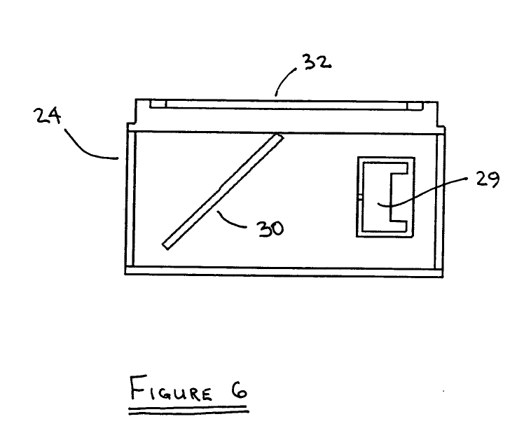

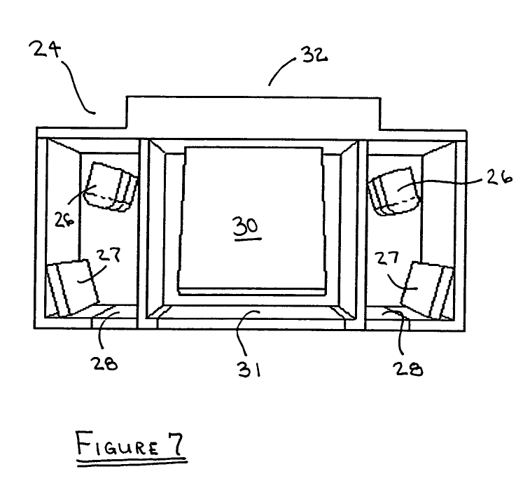

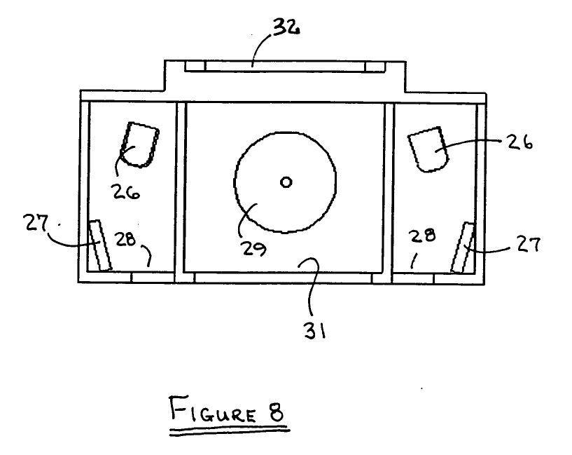

Referring now to FIGS. 4 through 8, an alternate embodiment of the portable vein locating device 10 is shown wherein the device 10 is implemented with a locating box 24, generally more bulky than the locating stick 11, but including a two dimensional liquid crystal display (“LCD”) 32 for increased display of a detected vein and a plurality of infrared LEDs 26 for increased transmission through the skin of infrared light. As shown in the figures, one or more infrared illuminators 25 is provided for transmission of infrared light through the person's skin where the infrared light is either absorbed by veins or partially backscattered as previously discussed. In the case of the locating box 24, however, backscattered infrared light is passed through a generally translucent imaging window 31 through the central portion of the base of the box 24 and imaged by a provided camera 29, which may be implemented using well known CCD technology or the like. To facilitate imaging, a mirror 30 may be positioned within the locating box 24 as particularly shown in FIGS. 5 through 7. In any case, the image through the translucent window 31 as captured by the camera 29 is displayed on the LCD 32 identifying for the medical caregiver the precise layout of veins located beneath the patient's skin. As will be appreciated by those of ordinary skill in the art, appropriate electronic circuitry (well within the level of ordinary skill in the art) is provided for operation of the camera 29 and display on the LCD 32 of the acquired image.

In the implementation comprising the locating box 24, the infrared illuminators 35 may comprise a plurality of LEDs 26 positioned within the locating box 24 and oriented to transmit infrared light through windows 28 through the outer portions of the base of the box 24. To facilitate light transmission, a plurality of mirrors 27 or mirrored surfaces may be provided adjacent to the LEDs 26 and the windows 28.

While the foregoing description is exemplary of the preferred embodiment of the present invention, those of ordinary skill in the relevant arts will recognize the many variations, alterations, modifications, substitutions and the like as are readily possible, especially in light of this description, the accompanying drawings and claims drawn thereto. For example, the generally ink pen-shaped protective case 22 of the locating stick 11 of the preferred embodiment of the present invention may be provided with an ink pen-style pocket clip 23 or the like for convenient carrying by a medical caregiver.

Likewise, those of ordinary skill in the art will recognize that appropriate circuitry may be implemented to modulate the infrared light passed through the skin at a predetermined frequency and to perform a synchronized signal amplification of the detected signal. In this manner, very high imaging quality may be obtained and the device may be readily utilized even in bright sunlight. In any case, because the scope of the present invention is much broader than any particular embodiment, the foregoing detailed description should not be construed as a limitation of the scope of the present invention, which is limited only by the claims appended hereto.

What is claimed is:

1. A portable vein locating device for determining the position beneath a person's skin of a vein, said vein locating device comprising:
an infrared illuminator for transmission through the person's skin of infrared light;
an infrared light detection array for detecting the presence of infrared light reflected from tissues beneath the person's skin; and
an indicator, said indicator being adapted to represent to the position beneath the person's skin of a vein according to the detection of infrared light by said detection array.

2. The portable vein locating device as recited in claim 1, wherein said infrared illuminator comprises an infrared light emitting diode.

3. The portable vein locating device as recited in claim 2, wherein said infrared illuminator further comprises a diffusing light guide.

4. The portable vein locating device as recited in claim 1, wherein said infrared illuminator comprises a diffusing light guide.

5. The portable vein locating device as recited in claim 1, wherein said infrared light detection array comprises an array of phototransistors.

6. The portable vein locating device as recited in claim 1, wherein said infrared light detection array comprises a charge coupled device sensor.

7. The portable vein locating device as recited in claim 6, wherein said sensor comprises a camera.

8. The portable vein locating device as recited in claim 1, wherein said infrared light detection array comprises a camera.

9. The portable vein locating device as recited in claim 8, wherein said indicator comprises a liquid crystal display.

10. The portable vein locating device as recited in claim 9, wherein said indicator is two dimensional.

11. The portable vein locating device as recited in claim 1, wherein said indicator comprises a plurality of light emitting diodes.

12. The portable vein locating device as recited in claim 11, wherein said light emitting diodes are arranged in a generally linear array.
